(19) United States
(12) Patent Application Publication
(10) Pub. No.: US 2025/0367356 A1
(43) Pub. Date: Dec. 4, 2025

(54) MEDICAL TREATMENT SUPPORT APPARATUS

(71) Applicant: TERUMO KABUSHIKI KAISHA, Tokyo (JP)

(72) Inventors: Yoshihito MACHIDA, Sagamihara Kanagawa (JP); Masaru IKEGAMI, Fujisawa Kanagawa (JP); Shotaro TERANE, Naka Kanagawa (JP); Ayato SUZUKI, Zama Kanagawa (JP)

(21) Appl. No.: 19/308,078

(22) Filed: Aug. 22, 2025

Related U.S. Application Data
(63) Continuation of application No. PCT/JP2024/003405, filed on Feb. 2, 2024.

(30) Foreign Application Priority Data
Feb. 28, 2023 (JP) 2023-030042

(51) Int. Cl. A61M 1/36; A61M 5/172; G16H 10/40; G16H 20/10

(52) U.S. Cl. CPC A61M 1/3672; A61M 5/1723; G16H 10/40; G16H 20/10; A61M 2205/3334; A61M 2205/3365; A61M 2205/502; A61M 2230/20

(57) ABSTRACT

A medical treatment support apparatus includes an interface circuit connectable to an extracorporeal blood circulation device and a medication device for administering a medicine to a patient and a processor configured to acquire administration information regarding administration of the medicine to the patient, acquire device operation information including values related to an operation of the device, execute a call to a machine learning model with the administration and device operation information to determine a test value that is expected from a blood test performed on the patient at a predetermined time after the medicine is administered, determine a dose of the medicine to be administered to the patient based on the determined test value, and control the medication device to administer the determined dose of the medicine to the patient.

### CROSS-REFERENCE TO RELATED APPLICATION

This application is a continuation of International Patent Application No. PCT/JP2024/003405 filed Feb. 2, 2024, which is based upon and claims the benefit of priority from Japanese Patent Application No. 2023-030042, filed Feb. 28, 2023, the entire contents of which are incorporated herein by reference.

### TECHNICAL FIELD

Embodiments of the present disclosure relate to a medical treatment support apparatus.

### BACKGROUND ART

There is a known blood circulation system that includes a blood circulation path connected to a patient and a centrifugal pump that circulates blood between the patient's body and a heart-lung machine, and adjusts the pressure of the blood by controlling the rotational speed of the centrifugal pump on the basis of a pressure measurement value obtained from a blood pressure measurement device.

For example, in an intensive care unit (ICU), an anticoagulant such as heparin is used to prevent a thrombus of a patient. The dosage of the anticoagulant can be determined on the basis of the weight of the patient, the experience of a doctor, and the like. In a case where the anticoagulant is excessively administered, the risk of developing a bleeding complication increases, and in a case where the anticoagulant is insufficient, the risk of developing a complication due to a thrombus increases. Furthermore, in a case where an extracorporeal blood circulation device, so-called ECMO (Extracorporeal Membrane Oxygenation), is used for a patient, it is difficult to manage the dose of the anticoagulant for the patient.

### SUMMARY

Embodiments of the present disclosure provide a medical treatment support apparatus that can accurately predict a test value of a blood test for a patient who is using an extracorporeal blood circulation device.

A medical treatment support apparatus comprises an interface circuit connectable to an extracorporeal blood circulation device and a medication device for administering a medicine to a patient; a memory that stores a program; and a processor configured to execute the program to: acquire administration information regarding administration of the medicine to the patient, acquire device operation information including one or more values related to an operation of the extracorporeal blood circulation device, execute a call to a machine learning model with the administration information and the device operation information to determine a test value that is expected from a blood test performed on the patient at a predetermined time after the medicine is administered, the machine learning model having been trained with: a plurality of pieces of administration information regarding administration of the medicine to different patients, a plurality of pieces of device operation information used by the extracorporeal blood circulation device for treating the different patients, and test values obtained before and after administration of the medicine to the different patients, determine a dose of the medicine to be administered to the patient based on the determined test value, and control the medication device to administer the determined dose of the medicine to the patient.

According to an embodiment, it is possible to accurately predict of a test value of a blood test for a patient using an extracorporeal blood circulation device.

### BRIEF DESCRIPTION OF DRAWINGS

FIG. 1 is a schematic diagram for illustrating an overview of an information processing system for generating a learning model according to an embodiment.

FIG. 2 is a schematic diagram for illustrating an overview of the information processing system using the generated learning model.

FIG. 3 is a schematic diagram illustrating a configuration example of the learning model.

FIG. 4 is a block diagram illustrating a configuration example of an information processing device for generating the learning model.

FIG. 5 is a schematic diagram illustrating a configuration example of a patient database (DB).

FIG. 6 is a block diagram illustrating a configuration example of an information processing device for using the generated learning model.

FIG. 7 is a flowchart illustrating a procedure of learning model generation processing.

FIG. 8 is a flowchart illustrating an example of a procedure of test value prediction processing.

FIG. 9 is a flowchart illustrating an example of a procedure of control processing based on a test result.

FIG. 10 is a flowchart illustrating another example of a procedure of test value prediction processing.

FIG. 11 is a schematic diagram illustrating an example of a prediction result.

FIG. 12 is an enlarged view of a graph in FIG. 11.

### DESCRIPTION OF EMBODIMENTS

A specific example of an information processing system according to an embodiment of the present invention will be hereinbelow described with reference to the drawings. Note that the present invention is not limited by these examples but is disclosed in the claims, and is intended to include all changes within the meaning and scope equivalent to the claims.

### <System Overview>

An information processing system according to an embodiment is a medical treatment aid system that outputs a predicted value for a test value of a blood test after a lapse of a predetermined time such as one hour or three hours from administration of an anticoagulant such as heparin, for example, a blood coagulation activity index such as activated clotting time (ACT) or activated partial thromboplastin time (APTT), for a subject to whom the anticoagulant is administered and to whom an ECMO is connected, in an ICU or the like. The information processing system uses a learning model based on machine learning, so-called artificial intelligence (AI), to generate a predicted test value. Note that, in the description of this disclosure, a medicine administered to a patient is an anticoagulant, but the medicine is not limited thereto, and may be, for example, an antiplatelet agent or the like.

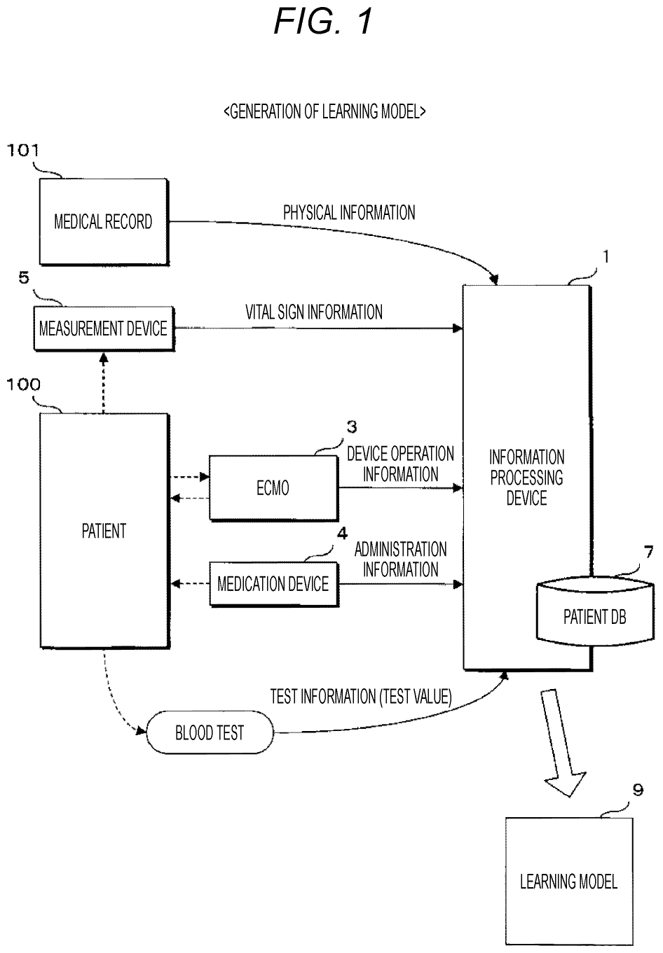

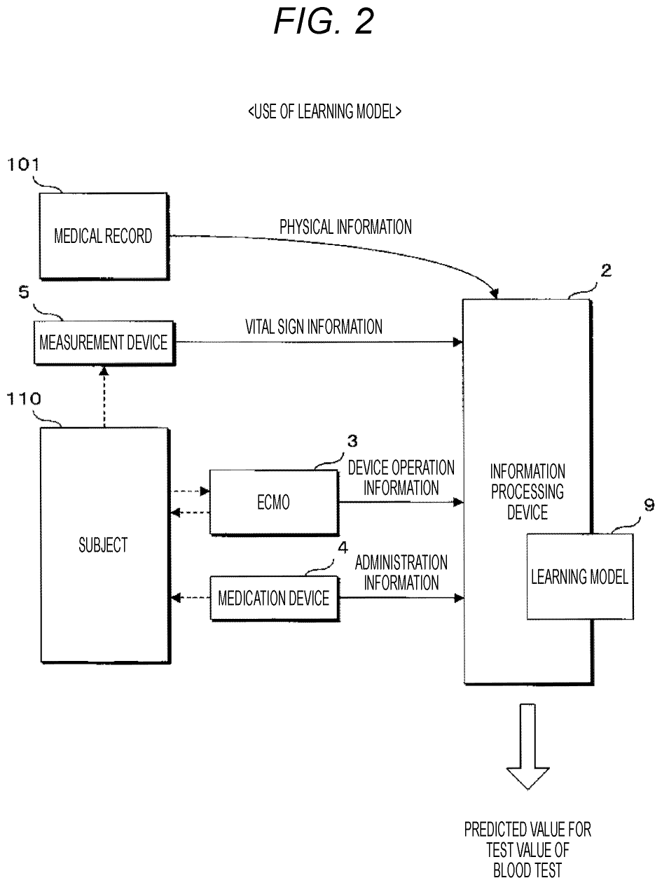

FIGS. 1 and 2 are schematic diagrams for illustrating an overview of the information processing system. FIG. 1 illustrates an overview of the information processing system in a stage of generating a learning model 9 that predicts the test value. FIG. 2 illustrates an overview of the information processing system in a stage of predicting the test value using the generated learning model 9. The information processing system includes information processing devices 1 and 2 as medical treatment support apparatuses, an ECMO 3, a medication device 4, a measurement device 5, and the like. Note that, in the information processing system, the information processing device that generates the learning model 9 in a stage of generating the learning model is the information processing device 1, and the information processing device that predicts the test value in a stage of using the learning model is the information processing device 2. However, the information processing device 1 and the information processing device 2 may be the same device.

In an embodiment, a patient 100 to be treated in an ICU or the like is provided with the ECMO 3, whereby extracorporeal circulation of blood is performed, and an anticoagulant such as heparin is administered by the medication device 4. The ECMO 3 is a device that takes blood from the patient 100, gives oxygen, and sends the blood back into the body of the patient 100. The medication device 4 is a device that automatically administers a drug to the patient 100 according to a particular dose, an administration cycle, and the like set by a user such as a doctor or a nurse. In addition, the measurement device 5 that measures vital signs such as blood pressure and heart rate as vital sign information is attached to the patient 100, and for example, a doctor, a nurse, or the like can monitor the measurement result of the measurement device 5 in real time. In addition, for the patient 100 to which the anticoagulant has been administered, for example, a blood test is performed at a predetermined cycle such as once per hour or once per three hours, and the test value such as ACT or APTT is obtained. Note that the vital sign information is information regarding a sign indicating that a human is in a living state, and includes information regarding blood pressure, pulse, body temperature, and respiration rate.

The information processing device 1 acquires these various types of information regarding the patient 100, stores the information in a patient database (DB) 7, and accumulates the information. In an embodiment, the information processing device 1 acquires at least device operation information regarding the operation of the ECMO 3, administration information regarding the anticoagulant administered to the patient 100, physical information of the patient 100 recorded in a medical record 101 or the like created by a doctor or the like, vital sign information of the patient 100 measured by the measurement device 5, and test information including the test value of the blood test of the patient 100, and stores the acquired information in the patient DB 7 in association with, for example, identification information of the patient 100.

The information processing device 1 acquires, for example, information such as the flow rate of blood in the extracorporeal blood circulation circuit of the ECMO 3 or the rotation speed of the pump as the device operation information regarding the operation of the ECMO 3, and stores the information in the patient DB 7. Furthermore, the device operation information acquired and stored by the information processing device 1 may include information regarding an operation range of the ECMO 3, for example, an upper limit value and a lower limit value of a setting value that can be set as the rotation speed of the pump of the ECMO 3. For example, the information processing device 1 may acquire the device operation information from the ECMO 3 by performing communication with the ECMO 3 via a network, or may receive input of a setting value from the user such as the doctor or the nurse who has set the operation of the ECMO 3 and acquire the setting value as the device operation information.

In addition, the information processing device 1 acquires, for example, information regarding a dose and an administration cycle of heparin as the administration information of the anticoagulant to the patient 100, and stores the acquired information in the patient DB 7. For example, the information processing device 1 can acquire the administration information of an anticoagulant from the medication device 4 by communicating with the medication device 4 via a network. In addition, for example, the information processing device 1 may receive input of a setting value from the user such as the doctor or the nurse who has set the operation of the medication device 4 and acquire the setting value as the administration information. In addition, for example, the administration of the anticoagulant to the patient may be performed not by the medication device 4 but by drip infusion, injection, or the like by the user such as the doctor or the nurse. In this case, the information processing device 1 may receive input of information such as a dose from the user who performed the administration and acquire the information as the administration information.

Furthermore, the information processing device 1 acquires, for example, information regarding the weight, height, and age of the patient 100 as the physical information of the patient 100, and stores the acquired information in the patient DB 7. For example, the information processing device 1 can acquire the electronic medical record 101 created by a doctor in charge of the patient 100 from, for example, a database of a hospital or the like, and acquire information regarding the weight, the age, and the like of the patient 100 included in the medical record 101 as the physical information. In addition, for example, the information processing device 1 may receive input of information regarding the weight, the age, and the like of the patient 100 from the user such as the doctor or the nurse in charge of the patient 100 and acquire the information as the physical information.

Furthermore, the information processing device 1 acquires, for example, a test value such as ACT or APTT obtained as a result of the blood test of the patient 100 as the test information of the patient 100, and stores the test value in the patient DB 7. In an embodiment, the blood test of the patient 100 is separately performed by the user such as the doctor, the nurse, or a laboratory technician, for example. The information processing device 1 receives input of the test information including the test value of the blood test from such the user and stores the information in the patient DB 7.

Furthermore, the information processing device 1 acquires, for example, blood pressure, heart rate, or the like as the vital sign information of the patient 100, and stores the acquired information in the patient DB 7. In an embodiment, the information processing device 1 acquires the vital sign information of the patient 100 continuously measured by the measurement device 5 by means of communication or the like. However, a doctor, a nurse, or the like may measure the blood pressure, the heart rate, or the like of the patient 100 and input the measurement result into the information processing device 1. Note that, in an embodiment, the vital sign information is used for processes such as generation of the learning model 9 and prediction of the test value using the learning model 9, but embodiments of this disclosure are not limited thereto, and the vital sign information does not need to be used for these processes.

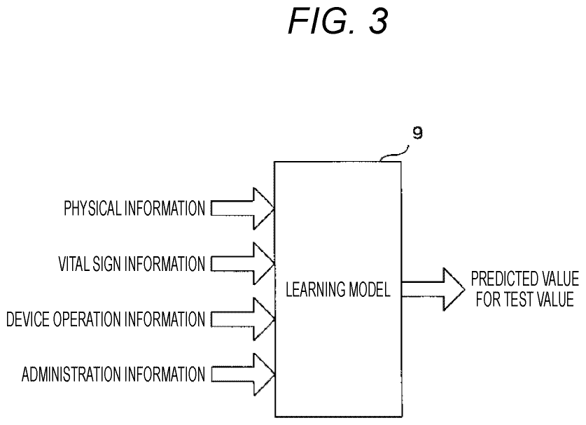

The information processing device 1 acquires the above-described device operation information, administration information, physical information, examination information, vital sign information, and the like for the plurality of (as many as possible) patients 100, stores the information in the patient DB 7, and performs machine learning processing of generating the learning model 9 using the stored information. FIG. 3 is a schematic diagram illustrating a configuration example of the learning model 9. The learning model 9 receives the physical information of the subject, the vital sign information of the subject, the device operation information regarding the operation of the ECMO 3 connected to the subject, and the administration information regarding the administration of the anticoagulant to the subject as input, and outputs the predicted value for the test value of the blood test of the subject after a lapse of a predetermined time. The information processing device 1 can generate the learning model 9 by generating learning data (or training data) in which the physical information, the vital sign information, the device operation information, and the administration information stored in the patient DB 7 used as input data are associated with the test value after a lapse of a predetermined time from administration of the anticoagulant used as output data (or true data), and performing processing of so-called supervised machine learning using the generated learning data. As the learning model 9, for example, learning models having various configurations such as a deep neural network (DNN), a support vector machine (SVM), logistic regression, or a decision tree can be adopted. Note that, the supervised machine learning processing for these learning models is an existing technology, so that detailed description thereof will be omitted.

The information processing device 1 transmits, to the information processing device 2 that uses the generated learning model 9, information regarding the learning model 9 generated in the machine learning processing, for example, information indicating the structure of the learning model 9 and information such as a value of an internal parameter determined by the machine learning. The information processing device 1 may transmit the information regarding the learning model 9 to the information processing device 2 by communication via a network, for example. Furthermore, the information regarding the learning model 9 may be manually transmitted and received from the information processing device 1 to the information processing device 2 via, for example, a recording medium by an administrator or the like of the information processing system. Furthermore, for example, a configuration in which the information processing device 1 that has generated the learning model 9 performs processing using the learning model 9 may be employed, that is, the information processing device 1 and the information processing device 2 may be the same device.

In addition, FIG. 2 illustrates an overview of the information processing system in a stage of predicting the test value using the pre-trained learning model 9. The information processing device 2 acquires and stores in advance information regarding the learning model 9 generated by the information processing device 1 by means of machine learning, and can perform prediction processing of the test value using the learning model 9. The information processing device 2 acquires physical information, vital sign information, device operation information, and administration information of a subject 110 to whom the anticoagulant is to be administered or has been administered, and inputs the acquired physical information, vital sign information, device operation information, and administration information into the learning model 9. The information processing device 2 acquires the predicted value for the test value after a lapse of a predetermined time from administration of the anticoagulant output by the learning model 9, displays the acquired predicted value on a display unit, and outputs a prediction result of the test value for the user such as the doctor or the nurse.

Note that the information processing device 2 may acquire the physical information, the vital sign information, the device operation information, and the administration information, for example, by communication with a device that manages these pieces of information, or may receive the information on the basis of input by the user such as the doctor or the nurse, for example.

Furthermore, in the information processing system, instead of predicting the test value on the basis of a certain value of the flow rate of blood, the rotation speed of the pump, or the like related to the operation of the ECMO 3 as the device operation information of the ECMO 3, the information processing device 2 may set an operation range such as an upper limit value and a lower limit value of the flow rate of blood, the rotation speed of the pump, or the like related to the operation of the ECMO 3, and based on the operation range, may determine an upper limit value and a lower limit value of the test value by determining the test value by changing the flow rate of blood in the ECMO 3 circuit, the rotation speed of the pump, or the like within the operation range. Accordingly, for example, in a case where the ECMO 3 is automatically operated within the operation range set by the user such as the doctor or the nurse, the information processing device 2 can predict the range of the test result of the subject 110 after a lapse of a predetermined time from the administration of the anticoagulant, and it is possible to expect that the user can determine whether the setting of the automatic operation of the ECMO 3 is appropriate on the basis of whether or not the range is within the appropriate range.

Similarly, instead of determining the test value on the basis of a certain value of a dose, an administration cycle, or the like of the anticoagulant for the subject 110 as the administration information of the anticoagulant, the information processing device 2 may determine a range of the test value such as an upper limit value and a lower limit value of the test value on the basis of an operation range such as an upper limit value and a lower limit value of the dose of the anticoagulant by the medication device 4, for example. Accordingly, for example, in a case where the medication device 4 is automatically operated within the operation range set by the user such as the doctor or the nurse, the information processing device 2 can predict the range of the test result of the subject 110 after a lapse of a predetermined time from the administration of the anticoagulant, and it is possible to expect that the user can determine whether or not the setting of the automatic operation of the medication device 4 is appropriate, or whether the dose of the anticoagulant is appropriate, on the basis of whether or not the range is within the appropriate range.

### <Device Configuration>

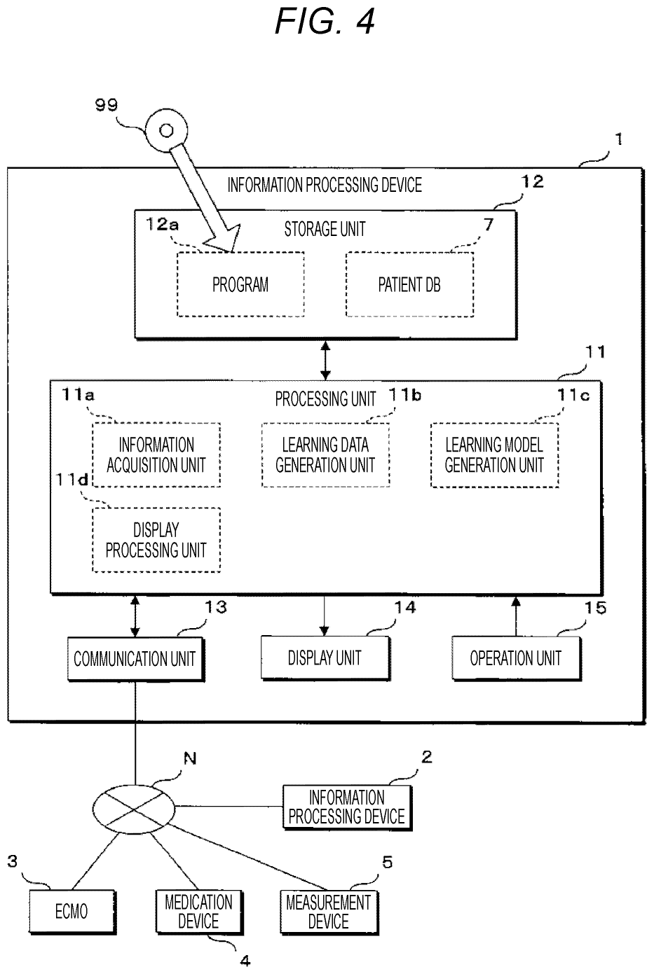

FIG. 4 is a block diagram illustrating a configuration example of the information processing device 1. The information processing device 1 collects information such as physical information, device operation information, administration information, and test information of the patient 100 and performs processing of generating the learning model 9 on the basis of the collected information, and can include a general-purpose computer such as a personal computer (PC) or a server computer. The information processing device 1 includes a processing unit 11, a storage unit 12, a communication unit 13, a display unit 14, an operation unit 15, and the like. Note that the description will be given assuming that the processing is performed by one information processing device 1, but a plurality of devices may perform the processing of the information processing device 1 in a distributed manner.

The processing unit 11 includes: a processor or a processing circuit such as a central processing unit (CPU), a micro-processing unit (MPU), a graphics processing unit (GPU), or a quantum processor, a read only memory (ROM), a random access memory (RAM), and the like. The processing unit 11 reads and executes a program 12a stored in the storage unit 12 to perform various processes such as a process of acquiring and collecting the physical information, the vital sign information, the device operation information, the administration information, the test information, and the like regarding the patient 100, and a process of generating a learning model for predicting a test value on the basis of these pieces of information.

The storage unit 12 includes, for example, a large-capacity storage device such as a hard disk. The storage unit 12 stores various programs executed by the processing unit 11 and various data required for processing of the processing unit 11. The storage unit 12 stores the program 12a executed by the processing unit 11. In addition, the storage unit 12 stores the patient DB 7 that stores and accumulates information such as physical information, vital sign information, device operation information, administration information, and test information for a plurality of different patients 100.

In an embodiment, the program (computer program or program product) 12a is recorded in a recording medium 99 such as a memory card or an optical disk, and the information processing device 1 reads the program 12a from the recording medium 99 and stores the same in the storage unit 12. Note that, the program 12a may be written in the storage unit 12 at a manufacturing stage of the information processing device 1, for example. In addition, for example, the information processing device 1 may acquire the program 12a distributed by a remote server device and the like by communication. For example, the program 12a recorded in the recording medium 99 may be read by a writing device, and written in the storage unit 12 of the information processing device 1. The program 12a may be provided in a form of distribution via a network, or may be provided in a form of being recorded in the recording medium 99.

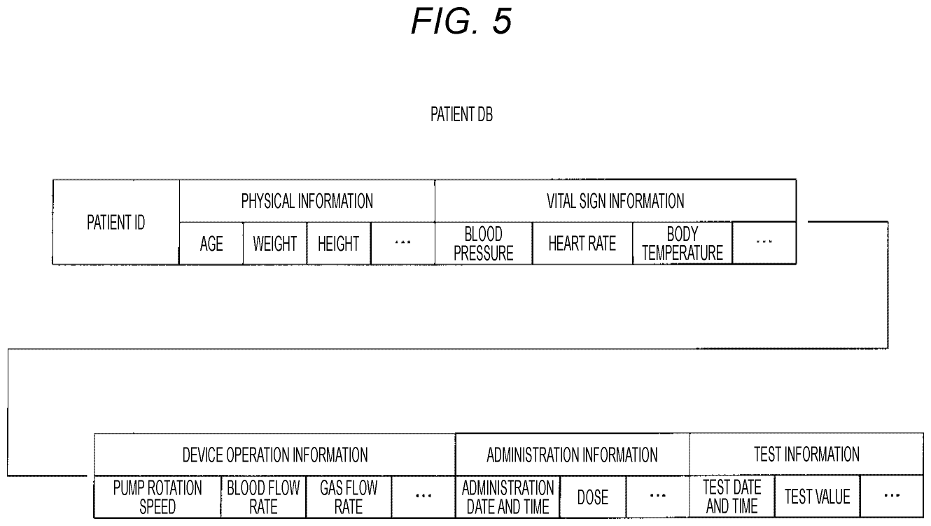

The patient DB 7 of the storage unit 12 is a database that stores information such as the physical information, the device operation information, the administration information, and the test information regarding the patients 100 in association with identification information such as IDs uniquely assigned to the patients 100, for example. FIG. 5 is a schematic diagram illustrating a configuration example of the patient DB 7. The patient DB 7 according to the present embodiment stores, for example, “patient ID”, “physical information”, “vital sign information”, “device operation information”, “administration information”, and “test information” in association with each other. The “patient ID” is identification information uniquely assigned to the patient 100, and may be information obtained by combining appropriate characters, numbers, and the like, and may be information such as a name of the patient.

The “physical information” may include, for example, information such as “age”, “weight”, and “height” of the patient 100. These pieces of information regarding the patient 100 may be acquired from an electronic medical record of the patient 100 stored in a database of a medical institution, for example, or may be acquired by receiving input from the user such as the doctor or the nurse in charge of the patient 100, for example. The “vital sign information” may include, for example, information such as “blood pressure”, “heart rate”, and “body temperature” of the patient 100. These pieces of information regarding the patient 100 are measured by the measurement device 5. The information processing device 1 may acquire these pieces of information by communication with the measurement device 5, or may receive input of a measurement result of the measurement device 5 from the user such as the doctor or the nurse and acquire these pieces of information.

In the present specification, “operation amount” of the extracorporeal blood circulation device refers to a parameter regarding the extracorporeal blood circulation device that can be changed, adjusted, or set and an amount regarding the extracorporeal blood circulation device that changes by changing the parameter. The “operation amount” of the extracorporeal blood circulation device may include a pump rotation speed of the extracorporeal blood circulation device, a blood flow rate in a circuit of the extracorporeal blood circulation device, and “gas flow rate” of a mixed gas of oxygen and air or the like in a device that exchanges gas with blood.

In the present specification, the “device operation information” of the extracorporeal blood circulation device includes information regarding an operation amount of the extracorporeal blood circulation device and a range of the operation amount. The “device operation information” may include, for example, information such as “pump rotation speed” related to the operation of the ECMO 3, “blood flow rate” in the circuit of the ECMO 3, and “gas flow rate” of a mixed gas of oxygen and air in a device that exchanges gas with blood, and an upper limit value or a lower limit value of the pump rotation speed of the ECMO 3. The information processing device 1 may acquire the information such as the “pump rotation speed”, the “blood flow rate”, and the “gas flow rate” by acquiring an operation history or the like from the ECMO 3 by communication with the ECMO 3 via a network, for example. Furthermore, the information processing device 1 may receive input of the information such as the “pump rotation speed”, the “blood flow rate”, and the “gas flow rate” from the user such as the doctor or the nurse who has performed operation setting of the ECMO 3, and store the information in the patient DB 7. Note that the illustrated patient DB 7 stores a plurality of pieces of information such as the “pump rotation speed”, the “blood flow rate”, and the “gas flow rate” as the “device operation information”, but the present invention is not limited thereto, and has only to store at least one piece of information out of the “pump rotation speed”, the “blood flow rate”, the “gas flow rate”, and the like.

The “administration information” may include, for example, information such as “administration date and time” and “dose” of the anticoagulant to the patient 100. The “administration date and time” is the date and time when the anticoagulant was administered to the patient 100, and the “dose” is the amount of the anticoagulant administered at this time. In a case where administration is performed to the patient 100 a plurality of times, information for the plurality of times can be stored in the “administration information”. The information processing device 1 may acquire the information such as the “administration date and time” and the “dose” by acquiring an operation history or the like from the medication device 4 by communication with the medication device 4 via a network, for example. In addition, the information processing device 1 may receive input of the information such as the “administration date and time” and the “dose” from the user who has performed operation setting of the medication device 4, the user who has administered the anticoagulant without using the medication device 4, or the like, and store the information in the patient DB 7. Although the illustrated patient DB 7 stores the information regarding the “dose” as the “administration information”, the present invention is not limited thereto, and information such as “administration cycle” or “administration rate” may be stored together with the “dose” or instead of the “dose”.

The “test information” may include, for example, information such as “test date and time” and “test value” regarding the blood test performed on the patient 100. The “test date and time” is the date and time when the blood test was performed on the patient 100, and the “test value” is a value such as ACT or APTT obtained as a result of the blood test. In a case where the test is performed on the patient 100 a plurality of times, information regarding the test for the plurality of times can be stored in the “test information”. The information processing device 1 receives input of the information such as the “test date and time” and the “test value” from the user who has performed the test, for example, and stores the information in the patient DB 7.

The communication unit 13 of the information processing device 1 is a network interface circuit that transmits and receives data to and from other devices such as the information processing device 2, the ECMO 3, the medication device 4, and the measurement device 5 via a network N such as a local area network (LAN) or the Internet. The communication unit 13 receives the device operation information from the ECMO 3, receives the administration information from the medication device 4, receives the vital sign information from the measurement device 5, and gives the received information to the processing unit 11. Furthermore, the communication unit 13 transmits information given from the processing unit 11, for example, information regarding the learning model 9 generated by machine learning, to the information processing device 2.

The display unit 14 includes a liquid crystal display and the like, and displays various images, characters, and the like on the basis of processing of the processing unit 11. The display unit 14 can display, for example, various types of information stored in the patient DB 7, information regarding the generated learning model 9, and the like.

The operation unit 15 receives operation from the user and notifies the processing unit 11 of the received operation. For example, the operation unit 15 receives the operation from the user via an input device such as a mechanical button or a touch panel and the like provided on a surface of the display unit 14. For example, the operation unit 15 may be an input device such as a mouse and a keyboard, and these input devices may be detachable from the information processing device 1.

Note that, the storage unit 12 may be an external storage device connected to the information processing device 1. The information processing device 1 may be a multi-computer including a plurality of computers or may be a virtual machine virtually constructed by software. Furthermore, the information processing device 1 is not limited to one having the above configuration, and does not need to include, for example, the display unit 14, the operation unit 15, and the like.

In the information processing device 1, the processing unit 11 reads and executes the program 12a stored in the storage unit 12, so that the functions of an information acquisition unit 11a, a learning data generation unit 11b, a learning model generation unit 11c, a display processing unit 11d, and the like are fulfilled by the processing unit 11 as software functional units.

The information acquisition unit 11a performs processing of acquiring the physical information, the vital sign information, the device operation information, the administration information, and the test information regarding the patient 100. The information acquisition unit 11a extracts and acquires information such as the age, the weight, and the height from the electronic medical record of the patient 100 stored in the database of the medical institution, for example, and stores these pieces of information in the patient DB 7 as the physical information. The information acquisition unit 11a acquires the measurement results of the blood pressure, the heart rate, the body temperature, and the like of the patient 100 by communicating with the measurement device 5 or receiving input of information from the user, for example, and stores these pieces of information in the patient DB 7 as the vital sign information. The information acquisition unit 11a acquires information such as the pump rotation speed, the blood flow rate, or the gas flow rate regarding the operation of the ECMO 3 connected to the patient 100 by communicating with the ECMO 3 or receiving input of information from the user, for example, and stores these pieces of information in the patient DB 7 as the device operation information. The information acquisition unit 11a acquires information such as the dose and the administration date and time of the anticoagulant administered to the patient 100 by communicating with the medication device 4 or receiving input of information from the user, for example, and stores the acquired information in the patient DB 7 as the administration information. In addition, the information acquisition unit 11a acquires information such as the test value and the test date and time of the blood test of the patient 100 by receiving input of information from the user who has performed the blood test of the patient 100, and stores the acquired information in the patient DB 7.

The learning data generation unit 11b performs processing of generating learning data for performing machine learning for generating the learning model 9 on the basis of the physical information, the vital sign information, the device operation information, the administration information, and the test information stored in the patient DB 7. For example, the learning data generation unit 11b can generate, as the learning data, information in which the weight of the patient 100 included in the physical information, the blood pressure included in the vital sign information, the pump rotation speed of the ECMO 3 included in the device operation information, the dose of the anticoagulant included in the administration information, and the test value included in the test information are associated with each other. At this time, the learning data generation unit 11b acquires the test value after a lapse of a predetermined time from the administration of the anticoagulant to the patient 100 from the patient DB 7 on the basis of the administration date and time included in the administration information and the test date and time included in the test information, and includes the test value in the learning data. The learning data generation unit 11b repeatedly performs similar processing for a plurality of patients stored in the patient DB 7 to generate a plurality of pieces of learning data. The learning data generation unit 11b stores the generated learning data in the storage unit 12.

The learning model generation unit 11c performs processing of generating the learning model 9 by performing machine learning using the plurality of pieces of learning data (or training data) generated by the learning data generation unit 11b. The learning model 9 is configured to receive physical information, vital sign information, device operation information, and administration information as input, and output a predicted value for a test value after a lapse of a predetermined time. The learning data generated by the learning data generation unit 11b is data in which the physical information, the vital sign information, the device operation information, the administration information, and the test information are associated with each other. Among these pieces of information, the physical information, the vital sign information, the device operation information, and the administration information correspond to input data into the learning model 9, and the test information corresponds to a true value of output data from the learning model 9. The learning model generation unit 11c performs processing of so-called supervised machine learning using the learning data and determines an internal parameter of the learning model 9 to generate the learning model 9. The learning model generation unit 11c may store information regarding the generated learning model in the storage unit 12 and transmit the information to the information processing device 2 by communication via the network N.

Note that the learning model 9 generated by the learning model generation unit 11c described above is a regression model that outputs the predicted value for the test information, but is not limited thereto, and the learning model generation unit 11c may generate the learning model 9 which is a classification model. The learning model 9 as the classification model receives physical information, vital sign information, device operation information, and administration information as input, and classifies, for example, test values after a lapse of a predetermined time into two classes of true and false. In addition, the learning model may classify the test value into other classes such as low, appropriate, and high. In this case, the learning data generation unit 11b determines which class the test value corresponds to on the basis of the test information stored in the patient DB 7, and generates the learning data in which the input data such as the physical information, the vital sign information, the device operation information, and the administration information is associated with the true class.

The display processing unit 11d performs processing of displaying various characters, images, and the like on the display unit 14. For example, in a case where the acquisition of various types of information by the information acquisition unit 11a is performed by receiving input from the user, the display processing unit 11d displays a screen for inputting these types of information on the display unit 14. Furthermore, for example, the display processing unit 11d may display information such as an evaluation value or a graph such as an accuracy rate or a matching rate regarding the learning model 9 generated by the learning model generation unit 11c.

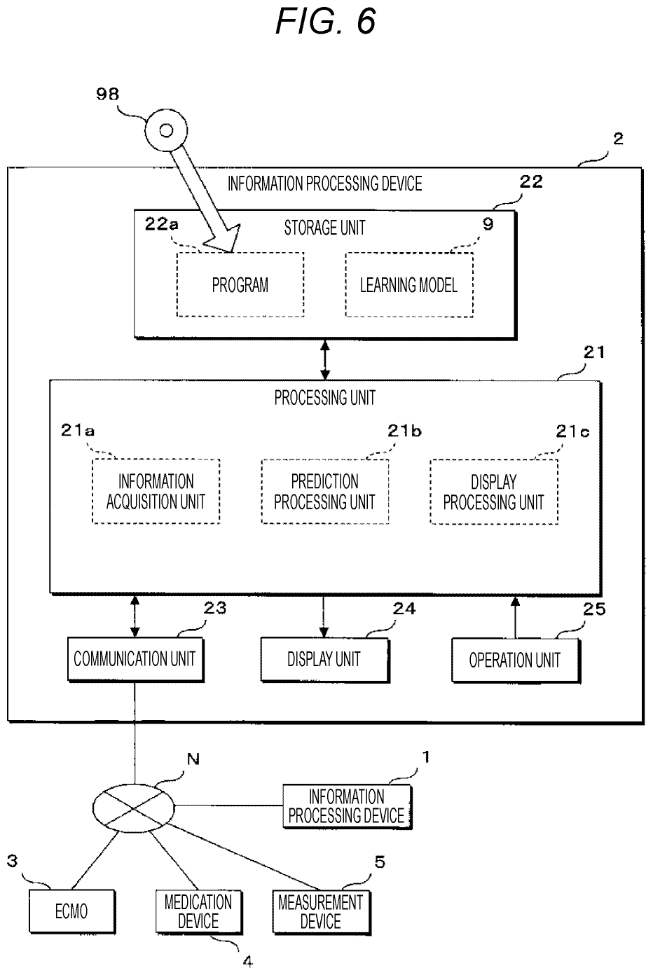

FIG. 6 is a block diagram illustrating a configuration example of the information processing device 2. The information processing device 2 is a device used by the user such as the doctor or the nurse in an ICT or the like of a medical institution, and can include, for example, a general-purpose computer such as a PC, a smartphone, or a tablet terminal device. The information processing device 2 includes a processing unit 21, a storage unit 22, a communication unit 23, a display unit 24, an operation unit 25, and the like.

The processing unit 21 includes: a processor such as a CPU, an MPU, a GPU, or a quantum processor, a ROM, a RAM, and the like. The processing unit 21 reads and executes a program 22a stored in the storage unit 22 to perform various processes such as a process of acquiring physical information, vital sign information, device operation information, and administration information regarding the subject 110, and a process of predicting a test value of a blood test of the subject 110 using the pre-trained learning model 9 on the basis of the acquired information. The storage unit 22 includes, for example, a large-capacity storage device such as a hard disk. The storage unit 22 stores the program 22a executed by the processing unit 21. Furthermore, the storage unit 22 stores the information regarding the learning model 9 generated by the information processing device 1.

In one embodiment, the program (computer program, or program product) 22a is recorded in a recording medium 98 such as a memory card or an optical disk, and the information processing device 2 reads the program 22a from the recording medium 98 and stores the same in the storage unit 22. Note that, the program 22a may be written in the storage unit 22 at a manufacturing stage of the information processing device 2, for example. In addition, for example, the information processing device 2 may acquire the program 22a distributed by a remote server device and the like by communication. For example, the program 22a recorded in the recording medium 98 may be read by a writing device, and written in the storage unit 22 of the information processing device 2. The program 22a may be provided in a form of distribution via a network, or may be provided in a form of being recorded in the recording medium 98.

The communication unit 23 is a network interface circuit that transmits and receives data to and from other devices such as the information processing device 1, the ECMO 3, the medication device 4, and the measurement device 5 via the network N. The communication unit 23 receives various types of data transmitted from another device and gives the data to the processing unit 21, and transmits data given from the processing unit 21 to another device. The display unit 24 includes a liquid crystal display or the like, and displays, for example, a prediction result for the test value of the blood test of the subject 110. The operation unit 25 receives operation of the user via an input device such as a mechanical button or a touch panel and the like provided on a surface of the display unit 24 and notifies the processing unit 21 of the received operation. For example, the operation unit 25 may be an input device such as a mouse and a keyboard, and these input devices may be detachable from the information processing device 2.

Note that, the storage unit 22 may be an external storage device connected to the information processing device 2. The information processing device 2 may be a multi-computer including a plurality of computers or may be a virtual machine virtually constructed by software. Furthermore, the information processing device 2 is not limited to one having the above configuration, and does not need to include, for example, the display unit 24, the operation unit 25, and the like.

In the information processing device 2, the processing unit 21 reads and executes the program 22a stored in the storage unit 22, so that the functions of an information acquisition unit 21a, a prediction processing unit 21b, a display processing unit 21c, and the like are fulfilled by the processing unit 21 as software functional units.

The information acquisition unit 21a performs processing of acquiring the physical information, the vital sign information, the device operation information, and the administration information regarding the subject 110. The information acquisition unit 21a can acquire these pieces of information by a similar method to that of the information acquisition unit 11a of the information processing device 1, for example. However, the information acquisition unit 21a may acquire the information by a different method from that of the information acquisition unit 11a of the information processing device 1.

The prediction processing unit 21b performs processing of predicting the test value of the blood test after a lapse of a predetermined time from the administration of the anticoagulant to the subject 110 based on the physical information, the vital sign information, the device operation information, and the administration information of the subject 110 acquired by the information acquisition unit 21a and the learning model 9 stored in the storage unit 22. For example, the prediction processing unit 21b inputs a body weight included in the physical information, blood pressure included in the vital sign information, pump rotation speed included in the device operation information, and a dose included in the administration information acquired regarding the subject 110 into the learning model 9. The prediction processing unit 21b acquires the predicted value for the test value output by the learning model 9 in response to the input of these pieces of information, thereby predicting the test value after a lapse of a predetermined time from the administration of the anticoagulant.

Furthermore, in the information processing system, instead of determining the test value on the basis of a certain value of, for example, the pump rotation speed as the device operation information of the ECMO 3, the prediction processing unit 21b may determine a range of the test value by predicting the test value per pump rotation speed by changing the pump rotation speed within an operation range such as an upper limit value and a lower limit value of the pump rotation speed. In this case, the information acquisition unit 21a acquires, as the device operation information, information such as an upper limit value and a lower limit value of the pump rotation speed that can be set for the ECMO 3, and an upper limit value and a lower limit value of the pump rotation speed that are set by the user for the ECMO 3. The prediction processing unit 21b can acquire a predicted value for a test value corresponding to the upper limit value in the operation range of the ECMO 3 and a predicted value for a test value corresponding to the lower limit value using the acquired information regarding the upper limit value and the lower limit value in the operation range and the learning model 9 stored in the storage unit 22, and set a range defined by the upper limit value and the lower limit value as a prediction result of the range of the test value.

Furthermore, in the information processing system, instead of determining the test value on the basis of a certain value of, for example, the dose of the anticoagulant as the administration information regarding the subject 110, the prediction processing unit 21b may determine the test value for each of a plurality of doses that are different in a stepwise manner by a predetermined amount, for example. In this case, the information acquisition unit 21a acquires, as the administration information, information regarding the upper limit value and the lower limit value of the dose of the anticoagulant to be administered to the subject 110 and the number of steps for prediction or the predetermined amount to make the dose different in a stepwise manner. The prediction processing unit 21b can acquire the predicted value for the test value using the learning model 9 for each of the plurality of doses, set the plurality of predicted values as the predicted results of the test values for the plurality of doses, create, for example, a graph associating the dose with the test value, and display the graph on the display unit 14.

Furthermore, the prediction processing unit 21b may classify the predicted value for the test value output from the learning model 9 into a plurality of classes on the basis of one or a plurality of set thresholds, and provide the user with the prediction result as classification. For example, the prediction processing unit 21b can set the prediction result as “low” in a case where the value of APTT output by the learning model 9 is lower than a first threshold value, set the prediction result as “appropriate” in a case where the value is between the first threshold value and a second threshold value, and set the prediction result as “high” in a case where the value is higher than the second threshold value. Note that the number of classes for classification may be two or four or more. Furthermore, the threshold for performing the classification is set in advance by an administrator of the information processing system, a user who uses the information processing system, or the like, and the set value is stored in the storage unit 22.

In addition, in a case where the learning model 9 is a classification model, the learning model 9 can determine which one of three classes, “low”, “appropriate”, or “high”, the value of APTT is classified into without performing the threshold determination as described above. The learning model 9 as the classification model outputs, for example, three values corresponding to three classes of “low”, “appropriate”, and “high”, and the class corresponding to the highest value is the prediction result. The number of classes when the learning model 9 as the classification model performs classification is not limited to three classes as described above, and may be two classes or four or more classes.

The display processing unit 21c performs processing of displaying various characters, images, and the like on the display unit 24. The display processing unit 21c performs processing of displaying information regarding the prediction result of the test value by the prediction processing unit 21b on the display unit 24. The display processing unit 21c may display one value as the predicted test value, for example, may display a class name in a case where the predicted test value is classified into a plurality of classes, for example, may display a range of the test value corresponding to the operation range of the ECMO 3, for example, may display a graph in which the dose and the test value are associated with each other, for example, or may display the test result in various modes other than these. How to display the test result can be set by the user, for example.

### <Learning Model Generation Processing>

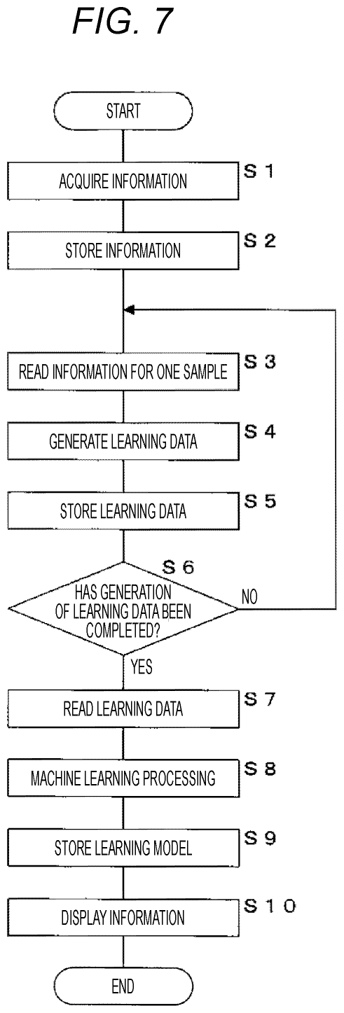

FIG. 7 is a flowchart illustrating a procedure of learning model generation processing performed by the information processing device 1. The information acquisition unit 11a of the processing unit 11 of the information processing device 1 acquires the physical information, the vital sign information, the device operation information, and the administration information regarding the patient 100 by communicating with the ECMO 3, the medication device 4, the measurement device 5, and the like, or by receiving input of information from the user (step S1). The information acquisition unit 11a stores the information acquired in step S1 in the patient DB 7 of the storage unit 12 in association with, for example, information such as the ID of the patient 100 (step S2).

The learning data generation unit 11b of the processing unit 11 reads information for one sample from among information regarding the plurality of patients 100 stored in the patient DB 7 (step S3). Based on the read information for one sample, the learning data generation unit 11b generates the learning data in which the weight of the patient 100 included in the physical information, the blood pressure included in the vital sign information, the pump rotation speed of the ECMO 3 included in the device operation information, the dose included in the administration information, and the test value after a lapse of a predetermined time from the administration of the anticoagulant included in the test information are associated with each other (step S4). The learning data generation unit 11b stores the learning data generated in step S4 in the storage unit 12 (step S5). The learning data generation unit 11b determines whether the generation of the learning data has been completed on the basis of, for example, whether the learning data has been generated for all the information stored in the patient DB 7 (step S6). In a case where the generation of the learning data has not been completed (S6: NO), the learning data generation unit 11b returns the processing to step S3 and repeats generation of the learning data on the basis of other information stored in the patient DB 7.

In a case where the generation of the learning data has been completed (S6: YES), the learning model generation unit 11c of the processing unit 11 reads the plurality of pieces of learning data generated by the learning data generation unit 11b from the storage unit 12 (step S7). The learning model generation unit 11c performs processing of supervised machine learning using the plurality of pieces of learning data read in step S7 on an untrained learning model for which a configuration or the like is determined in advance and an initial value is set in an internal parameter (step S8). The learning model generation unit 11c stores information regarding the learning model 9 generated by the machine learning processing in step S8, for example, information indicating the configuration of the learning model and information such as the determined internal parameter in the storage unit 12 (step S9). The display processing unit 11d of the processing unit 11 displays information regarding the accuracy and the like of the learning model 9 generated by the learning model generation unit 11c on the display unit 14 (step S10), and ends the processing.

The information regarding the learning model 9 generated in advance by the information processing device 1 is given to the information processing device 2 that performs the prediction processing of the test result. The information processing device 2 stores the given information regarding the learning model 9 in the storage unit 22, and uses the learning model 9 when predicting the test value of the blood test for the subject 110.

### <Test Value Prediction Processing 1>

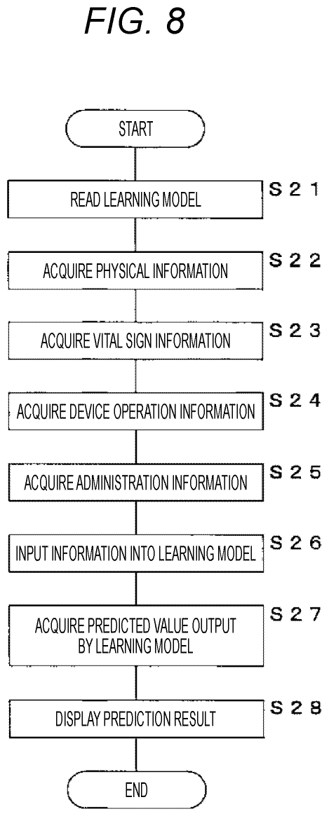

FIG. 8 is a flowchart illustrating an example of a procedure of test value prediction processing performed by the information processing device 2. The processing unit 21 of the information processing device 2 reads information regarding the learning model 9 stored in advance in the storage unit 22 (step S21).

Subsequently, the information acquisition unit 21a of the processing unit 21 acquires the physical information such as the weight of the subject 110 by, for example, extracting necessary information from the electronic medical record of the subject 110 or receiving input of information from the user (step S22). The information acquisition unit 21a acquires the vital sign information of the subject 110 by, for example, communicating with the measurement device 5 or receiving input of information from the user (step S23). The information acquisition unit 21a acquires the device operation information such as the pump rotation speed of the ECMO 3 connected to the subject 110 by, for example, acquiring information regarding the current operation by communicating with the ECMO 3 or receiving input of information from the user (step S24). The information acquisition unit 21a acquires the administration information such as the dose of the anticoagulant to the subject 110 by, for example, acquiring information such as the current dose by communicating with the medication device 4 or receiving input of information from the user (step S25).

Subsequently, the prediction processing unit 21b of the processing unit 21 inputs the physical information acquired in step S22, the vital sign information acquired in step S23, the device operation information acquired in step S24, and the administration information acquired in step S25 into the learning model 9 read in step S21 (step S26). The prediction processing unit 21b acquires the predicted value for the test value of the blood test after a lapse of a predetermined time from the administration of the anticoagulant to the subject 110, which is to be output by the learning model 9 in response to the input of these pieces of information (step S27). The display processing unit 21c of the processing unit 21 displays the predicted value acquired in step S27 on the display unit 24 as a prediction result (step S28), and ends the processing.

In the information processing system, the information processing device 2 can generate the predicted test result of the subject 110 using the learning model 9 generated in advance. The information processing device 2 may generate the test result by acquiring information input by the user such as the doctor or the nurse or may generate the test result by acquiring information by communication from a device such as the ECMO 3, the medication device 4, or the measurement device 5. In a case where the information processing device 2 performs prediction on the basis of information input from the user, for example, whether the pump rotation speed of the ECMO 3 and the dose of the anticoagulant determined by the user himself/herself are appropriate can be determined on the basis of the prediction result of the information processing device 2. In a case where the information processing device 2 acquires information from the device and performs prediction, for example, it is possible to periodically acquire information from the device and repeatedly perform prediction, and in a case where there is an abnormality in the prediction result, it is possible to warn the user or control the device on the basis of the prediction result.

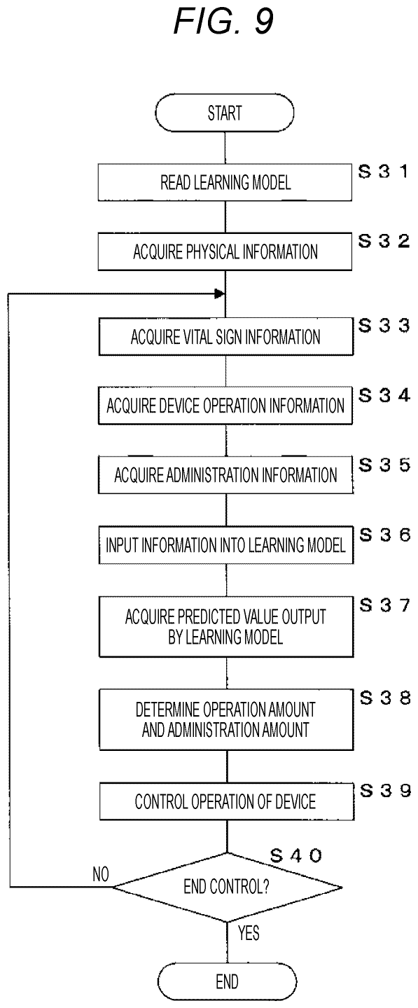

FIG. 9 is a flowchart illustrating an example of a procedure in a case where the information processing device 2 performs control processing based on the test result. The processing unit 21 of the information processing device 2 reads information regarding the learning model 9 stored in advance in the storage unit 22 (step S31). The information acquisition unit 21a of the processing unit 21 acquires the physical information such as the weight of the subject 110 by, for example, extracting necessary information from the electronic medical record of the subject 110 or receiving input of information from the user (step S32).

Subsequently, the information acquisition unit 21a communicates with the measurement device 5 through the communication unit 23, and acquires the vital sign information such as the current blood pressure of the subject 110 from the measurement device 5 (step S33). The information acquisition unit 21a communicates with the ECMO 3 through the communication unit 23, and acquires the device operation information such as the current pump rotation speed from the ECMO 3 (step S34). The information acquisition unit 21a communicates with the medication device 4 through the communication unit 23, and acquires the administration information such as the current dose from the medication device 4 (step S35). The prediction processing unit 21b of the processing unit 21 inputs the physical information acquired in step S32, the vital sign information acquired in step S33, the device operation information acquired in step S34, and the administration information acquired in step S35 into the learning model 9 read in step S31 (step S36). The prediction processing unit 21b acquires the predicted value for the test value of the blood test after a lapse of a predetermined time from the administration of the anticoagulant to the subject 110, which is to be output by the learning model 9 in response to the input of these pieces of information (step S37).

Subsequently, the processing unit 21 determines the operation amount of the ECMO 3 and the administration amount of the anticoagulant by the medication device 4 on the basis of the predicted value for the test value acquired from the learning model 9 in step S37 (step S38). For example, the information processing device 2 stores in advance a table in which a difference value between an appropriate value and a predicted value regarding the test value such as ACT or APTT is associated with an increase/decrease value of the operation amount of the ECMO 3 and an increase/decrease value of the dose by the medication device 4 according to the difference value. The processing unit 21 calculates a difference between the predicted value acquired in step S37 and the predetermined appropriate value, and acquires an increase/decrease value corresponding to the calculated difference value from the table. The processing unit 21 can determine the operation amount of the ECMO 3 by applying the increase/decrease value to the current operation amount, and can determine the dose of the medication device 4 by applying the increase/decrease value to the current dose. The test value may be predicted again on the basis of the determined dose to provide an opportunity to confirm whether the test value is an appropriate value. However, the method of determining the operation amount and the dose described above is an example and is not limited thereto, and the information processing device 2 may determine the operation amount of the ECMO 3 and the dose of the medication device 4 by any method on the basis of the prediction result of the test value.

The processing unit 21 controls the operations of the ECMO 3 and the medication device 4 on the basis of the operation amount of the ECMO 3 and the dose of the medication device 4 determined in step S38 (step S39). At this time, the processing unit 21 can control the operation of the ECMO 3, for example, by transmitting information regarding the determined operation amount to the ECMO 3 and giving a command to the ECMO 3 to perform the operation at this operation amount. Similarly, the processing unit 21 can control the operation of the medication device 4, for example, by transmitting information regarding the determined dose to the medication device 4 and giving a command to the medication device 4 to perform the medication at this dose.

The processing unit 21 determines whether to end the control of the ECMO 3 and the medication device 4 on the basis of, for example, whether an operation to end the control has been performed by the user (step S40). In a case where the control is not ended (S40: NO), the processing unit 21 returns the processing to step S33, newly acquires the vital sign information, the device operation information, and the administration information, and repeats the control processing. In a case where the control is ended (S40: YES), the processing unit 21 ends the control processing of the ECMO 3 and the medication device 4.

### <Test Value Prediction Processing 2>

In the above-described test value prediction processing 1, the information processing device 2 determines one predicted test value using the learning model 9 on the basis of a set of physical information, vital sign information, device operation information, and administration information. On the other hand, in test value prediction processing 2, the information processing device 2 determines a plurality of predicted test values on the basis of combinations of an operation range of the ECMO 3 and a setting step of the dose of the medication device 4.

For example, a user such as a doctor or a nurse inputs, into the information processing device 2, as the device operation information, an operation range defined by, for example, an upper limit value and a lower limit value of the operation amount such as the pump rotation speed or the blood flow rate of the ECMO 3 used for the subject 110. The device operation information of the ECMO 3 can be input as an operation range of an upper limit value of 5000 rpm and a lower limit value of 500 rpm, for example. Furthermore, the user inputs, into the information processing device 2, as the administration information, an operation range defined by, for example, an upper limit value and a lower limit value of the operation amount such as the dose of the medication device 4 used for the subject 110, and an increase/decrease amount that can change the setting of the operation amount. The administration information of the medication device 4 can be input as a combination of an operation range such as an upper limit value of 10000 units and a lower limit value of 1000 units, and an increase/decrease amount of 1000 units, for example. Note that each of the operation ranges of the device may be, for example, a range indicating a limit to which the device can operate, may be, for example, a range that can be set by the user for the device, or may be, for example, a range appropriately determined by the user within a range that can be set for the device. In addition, the operation range is not limited to one defined by the upper limit value and the lower limit value, and for example, may be defined only by the upper limit value, for example, may be defined only by the lower limit value, or may be defined on the basis of values other than these.

The information processing device 2 creates, for example, a plurality of combinations of pump rotation speeds of the ECMO 3 and doses of the medication device 4 on the basis of the input device operation information and administration information, and determines a predicted test value for each combination. For example, in a case where the upper limit value of 5000 rpm and the lower limit value of 500 rpm of the pump rotation speed are input as the device operation information, and the upper limit value of 7000 units, the lower limit value of 1000 units, and the increase/decrease amount of 1000 units of the dose are input as the administration information, 2 values of the pump rotation speed×7 values of the dose=14 combinations can be created. The information processing device 2 can obtain 14 predicted values by performing prediction using the learning model 9 for each of 14 combinations, for example. The information processing device 2 can present a prediction result to the user by, for example, generating a graph, a table, or the like on the basis of the plurality of predicted values obtained for the plurality of combinations of the device operation information and the administration information and displaying the graph, the table, or the like on the display unit 24.

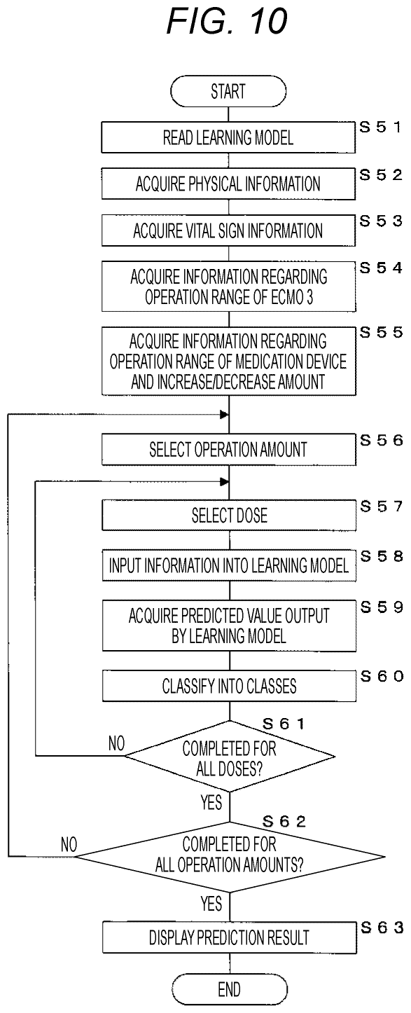

FIG. 10 is a flowchart illustrating another example of a procedure of test value prediction processing performed by the information processing device 2. The processing unit 21 of the information processing device 2 reads information regarding the learning model 9 stored in advance in the storage unit 22 (step S51). The information acquisition unit 21a of the processing unit 21 acquires the physical information such as the weight of the subject 110 by, for example, extracting necessary information from the electronic medical record of the subject 110 or receiving input of information from the user (step S52). In addition, the information acquisition unit 21a communicates with the measurement device 5 through the communication unit 23, and acquires the vital sign information such as the blood pressure of the subject 110 (step S53).

The information acquisition unit 21a acquires the operation range of the ECMO 3 connected to the subject 110 as the device operation information on the basis of, for example, input from the user (step S54). At this time, the information acquisition unit 21a displays a message prompting input of the operation range on the display unit 24, and accepts input of, for example, the upper limit value and the lower limit value as the operation range of the ECMO 3 from the user. However, in a case where the operation range of the ECMO 3 is not information changed frequently, the information processing device 2 may store, for example, the operation range previously received from the user in the storage unit 22 as set information, and the information acquisition unit 21a may acquire the operation range by acquiring the set information from the storage unit 22 in step S54.

In addition, the information acquisition unit 21a acquires the operation range of the medication device 4 that administers the anticoagulant to the subject 110 and the increase/decrease amount as the administration information on the basis of, for example, input from the user (step S55). At this time, the information acquisition unit 21a displays a message prompting input of the operation range and the increase/decrease amount on the display unit 24, and receives input of the upper limit value and the lower limit value of the operation range of the medication device 4 and the increase/decrease amount of the dose from the user. However, in a case where the operation range of the medication device 4 and the increase/decrease amount are not information changed frequently, the information processing device 2 may store, for example, the operation range and the increase/decrease amount previously received from the user in the storage unit 22 as set information, and the information acquisition unit 21a may acquire the operation range and the increase/decrease amount by acquiring the set information from the storage unit 22 in step S55.

Subsequently, the prediction processing unit 21b of the processing unit 21 selects one operation amount, for example, either the upper limit value or the lower limit value, from the operation range of the ECMO 3 acquired in step S54 (step S56). In addition, the prediction processing unit 21b selects one dose from among a plurality of doses that can be taken within the operation range on the basis of the operation range of the medication device 4 and the increase/decrease amount acquired in step S55 (step S57).

Subsequently, the prediction processing unit 21b of the processing unit 21 inputs the physical information acquired in step S52, the vital sign information acquired in step S53, the operation amount selected in step S56, and the information such as the dose selected in step S57 into the learning model 9 read in step S51 (step S58). The prediction processing unit 21b acquires the predicted value for the test value of the blood test after a lapse of a predetermined time from the administration of the anticoagulant to the subject 110, which is to be output by the learning model 9 in response to the input of these pieces of information (step S59). In addition, the prediction processing unit 21b classifies the predicted value acquired in step S59 into a plurality of classes by comparing the predicted value with one or a plurality of predetermined thresholds (step S60).

The prediction processing unit 21b determines whether the prediction using the learning model 9 has been completed for all the doses that can be selected in step S57 (step S61). In a case where the prediction has not been completed for all the doses (S61: NO), the prediction processing unit 21b returns the processing to step S57, selects another dose, and repeats the similar processing. In a case where the prediction has been completed for all the doses (S61: YES), the prediction processing unit 21b determines whether or the prediction using the learning model 9 has been completed for all the operation amounts that can be selected in step S56 (step S62). In a case where the prediction has not been completed for all the operation amounts (S62: NO), the prediction processing unit 21b returns the processing to step S56, selects another operation amount, and repeats the similar processing.

In a case where the prediction has been completed for all the operation amounts (S62: YES), the display processing unit 21c of the processing unit 21 generates and displays a screen for presenting the prediction result on the display unit 24 on the basis of the plurality of predicted values acquired in step S59, the result of classification performed in step S60, and the like (step S63), and ends the processing.

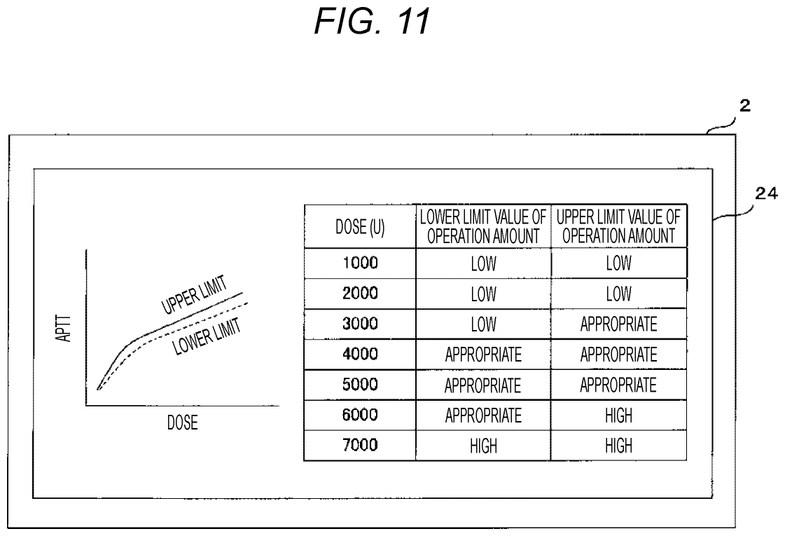

FIG. 11 is a schematic diagram illustrating an example of a prediction result generated and displayed by the information processing device 2 according to an embodiment. The illustrated example is a prediction result in a case where the information processing device 2 acquires 14 predicted values for APTT as test values on the basis of 14 combinations based on 2 values of the upper limit value and the lower limit value as the operation amount of the ECMO 3 and 7 doses that can be set in the medication device 4. In this example, it is assumed that the upper limit value is input as 7000 and the lower limit value is input as 1000 regarding the operation range of the medication device 4, and the increase/decrease amount is input as 1000, and there are seven doses of 1000, 2000, 3000, 4000, 5000, 6000, and 7000.

In the illustrated example, the information processing device 2 generates and displays a graph of the prediction result in the left region of the screen of the display unit 24, and generates and displays a table of the prediction result in the right region. In the graph of the prediction result, the horizontal axis represents the dose of the anticoagulant, and the vertical axis represents the predicted value for APTT. The graph line corresponding to the upper limit value of the operation amount of the ECMO 3 is indicated by a solid line, and the graph line corresponding to the lower limit value is indicated by a broken line. In addition, in the table of the prediction result, 14 predicted values based on 7 doses and 2 operation amounts are classified into three classes of “low”, “appropriate”, and “high”, and the prediction results are arranged in a matrix form of 2×7. Based on the illustrated prediction result, the user can determine the dose of the anticoagulant for the subject 110, for example. Furthermore, for example, the user can determine that the predicted value is “appropriate” when the dose is 4000 units or 5000 units, regardless of whether the pump rotation of the ECMO 3 is the lower limit value or the upper limit value, on the basis of the table illustrated in FIG. 11.

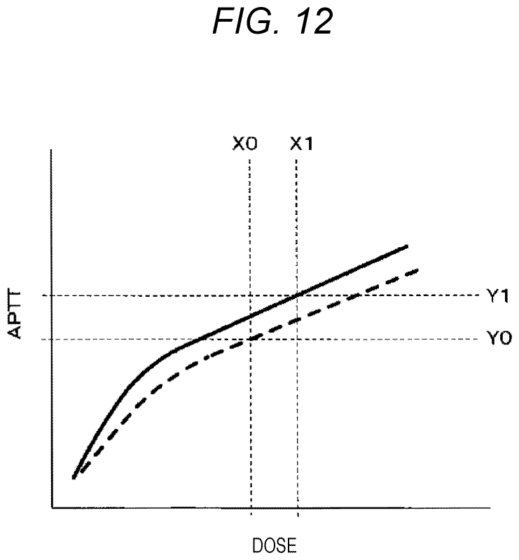

FIG. 12 is an enlarged view of the graph in FIG. 11. For example, it is assumed that the ECMO 3 is automatically operated within the range from the upper limit value to the lower limit value of the operation amount such as the pump rotation speed. The ECMO 3 can measure, for example, arterial oxygen saturation (SpO2), mixed venous oxygen saturation (SvO2), arterial oxygen partial pressure (PaO2), arterial carbon dioxide partial pressure (PaCO2), circuit internal pressure, or the like of the subject 110, and feedback-control the operation amount such as the pump rotation speed within the predetermined range from the upper limit value to the lower limit value. Here, for example, in a case where the appropriate range of APTT for the subject 110 is the range from Y0 to Y1 indicated by horizontal broken lines in FIG. 12, it can be determined that the dose of the anticoagulant to be administered to the subject 110 is the range from X0 to X1 indicated by vertical broken lines in FIG. 12.

Based on this determination, the user sets the administration amount of the anticoagulant by the medication device 4 to an appropriate value in the range from X0 to X1 and performs automatic operation, so that it can be expected that APTT of the subject 110 after a lapse of a predetermined time falls within the appropriate range from Y0 to Y1. Note that the administration of the anticoagulant may be performed by the user with an appropriate dose from X0 to X1 using, for example, drip infusion, injection, or the like without using the medication device 4.

In the information processing system having the above configuration, the information processing device 1 acquires the learning data in which the administration information regarding administration of the anticoagulant to the patient 100 and the device operation information regarding the operation amount of the ECMO 3 connected to the patient 100 are associated with the test value related to the blood of the patient 100 after a lapse of a predetermined time from administration of the anticoagulant, and generates the learning model 9 by performing the supervised machine learning processing using the acquired learning data. The learning model 9 receives the administration information and the device operation information of the subject as input, and outputs the predicted value for the test value of the subject. By using information regarding the operation amount within the operation range of the ECMO 3 as the device operation information included in the learning data, the predicted value output by the learning model 9 can be within the operation range of the ECMO 3. By using the learning model 9 generated by the information processing device 1, it can be expected to achieve prediction of the test value related to the blood of the subject. In addition, even in a case where the ECMO is automatically operated and the pattern of the in-circuit flow rate or the pump rotation speed of the ECMO during treatment is not known in advance, a medical worker can learn about the range of the test value regarding the blood of the subject by predicting the range of the test value within the operation range of the ECMO. Therefore, the medical worker can appropriately determine the treatment policy for the subject and the setting of the ECMO, the medication device, and the like with reference to the information.

In the above-described information processing system, the information processing device 2 acquires the administration information regarding administration of the anticoagulant to the subject 110, acquires the device operation information including the operation amount of the ECMO 3 connected to the subject 110 or the range thereof, and determines the predicted test value corresponding to the operation amount or the test value within the range of the operation amount on the basis of the learning model 9 on which the machine learning has been performed in advance and the acquired administration information and device operation information. The learning model 9 receives the administration information of the subject 110 and the operation amount of the ECMO 3 connected to the subject 110 as input, and outputs the predicted value for the test value related to the blood of the subject 110 after a lapse of a predetermined time from administration of the anticoagulant. Accordingly, it can be expected that the information processing system achieves prediction of the test value related to the blood of the subject 110.

Furthermore, in the above-described information processing system, the information processing device 2 further acquires the physical information regarding the body of the subject 110, and determines the predicted test value on the basis of the learning model 9 on which machine learning is performed in advance and the acquired administration information, device operation information, and physical information. Accordingly, the information processing system can predict the test value in consideration of a difference in physical information such as the weight, height, or age of the subject, and improvement in prediction accuracy can be expected.

Furthermore, in the above-described information processing system, the information processing device 2 determines the administration amount of the anticoagulant or the operation amount of the ECMO 3 on the basis of the predicted value for the test value of the subject 110, and controls the operation of the medication device 4 or the ECMO 3 on the basis of these determined amounts. Accordingly, it can be expected that the information processing system achieves the automatic operation of the ECMO 3 or the medication device 4 reflecting the prediction result of the learning model 9.

Furthermore, in the above-described information processing system, the information processing device 2 classifies the predicted value of the learning model 9 into the plurality of classes such as “low”, “appropriate”, and “high”. Accordingly, the information processing system can provide the prediction result of the test value by the learning model 9 not as a simple predicted value of the test value but as a plurality of classes into which the predicted value is classified, and thus it can be expected that the user can easily grasp the prediction result. Furthermore, the information processing device 2 may receive, from the user, a setting of a threshold for comparison with the predicted value regarding the classification. Accordingly, it can be expected that the information processing system classifies the predicted value under a condition suitable for the user.

Furthermore, in the above-described information processing system, the information processing device 2 determines the predicted test values using the learning model 9 for a plurality of combinations of the dose of the anticoagulant and the operation amount of the ECMO 3, and outputs the correspondence relationship among the plurality of doses, operation amounts, and the predicted values as a graph or a table. As for the plurality of combinations, the predicted values of the test results corresponding to the upper limit value, the lower limit value, the median value, or the like of the operation amount of the ECMO 3 may be included. Accordingly, it can be expected that the information processing system causes the user to grasp how the prediction result changes in the operation range of the ECMO 3 or the operation range of the medication device 4, for example.

Note that the case of handling an anticoagulant such as heparin as a medicine to be administered to the patient 100 and the subject 110 has been described, but the medicine is not limited thereto, and the medicine may be, for example, an antiplatelet medicine or various medicines that may affect the flow of blood. In addition, although the pump rotation speed has been described as an example of the operation amount of the ECMO 3, the operation amount of the ECMO 3 is not limited thereto, and may be, for example, a blood flow rate or a gas flow rate, and various other amounts can be adopted as the operation amount. In addition, although ACT or APTT has been described as an example of the test value related to the test of blood, the test value is not limited thereto, and any value may be adopted as the test value.

In addition, the learning model 9 receives the physical information, the vital sign information, the device operation information, and the administration information as input, and outputs the predicted value for the test value. However, the present disclosure is not limited to this configuration. The learning model 9 may be configured to receive at least the device operation information and the administration information as input, and not to receive the physical information and/or the vital sign information as input. In this case, the information processing devices 1 and 2 do not need to acquire the physical information and/or the vital sign information.

It should be construed that the embodiments disclosed herein are illustrative in all respects rather than restrictive. The scope of the present disclosure is defined not by the meanings described above but by the claims, and is intended to include meanings equivalent to the claims and all modifications within the scope.

Some or all of the subject matters described in the respective embodiments can be combined together. In addition, some or all of the independent claims and their dependent claims described in the claims can be combined together, regardless of their dependent relationships. Furthermore, although a form (multiple dependent claim form) in which a claim dependent on two or more other claims is described is used in the claims, the claim form is not limited thereto. The present invention may be described by using a form in which a multiple dependent claim dependent on at least one multiple dependent claim is described.

What is claimed is:

1. A medical treatment support apparatus comprising:
an interface circuit connectable to an extracorporeal blood circulation device and a medication device for administering a medicine to a patient;
a memory that stores a program; and
a processor configured to execute the program to:
acquire administration information regarding administration of the medicine to the patient,
acquire device operation information including one or more values related to an operation of the extracorporeal blood circulation device,
execute a call to a machine learning model with the administration information and the device operation information to determine a test value that is expected from a blood test performed on the patient at a predetermined time after the medicine is administered, the machine learning model having been trained with:
a plurality of pieces of administration information regarding administration of the medicine to different patients,
a plurality of pieces of device operation information used by the extracorporeal blood circulation device for treating the different patients, and
test values obtained before and after administration of the medicine to the different patients,
determine a dose of the medicine to be administered to the patient based on the determined test value, and
control the medication device to administer the determined dose of the medicine to the patient.

2. The medical treatment support apparatus according to claim 1, wherein
the machine learning model has been further trained using physical information and vital sign information relating to the different patients, and
the processor executes the program to:
acquire physical information and vital sign information relating to the patient to which the extracorporeal blood circulation device is connected, and
execute the call to the machine learning model further using the administration information and the device operation information.

3. The medical treatment support apparatus according to claim 2, wherein
the interface circuit is connectable to a measurement device for measuring a vital sign of the patient, and
the vital sign information is acquired from the measurement device.

4. The medical treatment support apparatus according to claim 1, further comprising:
a display device, wherein
the processor executes the program to:
classify the determined test value into a plurality of classes using one or more threshold values,
generate a screen showing the determined dose and one of the classes corresponding to the determined test value, and
control the display device to display the screen.

5. The medical treatment support apparatus according to claim 1, wherein
the medicine is an anticoagulant or an antiplatelet agent.

6. The medical treatment support apparatus according to claim 1, wherein
the one or more values include a pump rotation speed or a blood flow rate of the extracorporeal blood circulation device.

7. The medical treatment support apparatus according to claim 1, wherein
the determined test value is activated clotting time (ACT) or activated partial thromboplastin time (APTT).

8. A medical treatment support apparatus comprising:
an interface circuit connectable to an extracorporeal blood circulation device connected to a patient;
a memory that stores a program; and
a processor configured to execute the program to:
acquire administration information regarding administration of a medicine to the patient,
acquire device operation information including one or more values related to an operation of the extracorporeal blood circulation device,
execute a call to a machine learning model with the administration information and the device operation information to determine a test value that is expected from a blood test performed on the patient at a predetermined time after the medicine is administered, the machine learning model having been trained with:
a plurality of pieces of administration information regarding administration of the medicine to different patients,
a plurality of pieces of device operation information used by the extracorporeal blood circulation device for treating the different patients, and
test values obtained before and after administration of the medicine to the different patients,
determine a value related to the operation and to be used for treating the patient based on the determined test value, and
control the extracorporeal blood circulation device according to the determined value.

9. The medical treatment support apparatus according to claim 8, wherein
the machine learning model has been further trained further using physical information and vital sign information relating to the different patients, and
the processor executes the program to:
acquire physical information and vital sign information relating to the patient to which the extracorporeal blood circulation device is connected, and
execute the call to the machine learning model further using the physical information and the vital sign information.

10. The medical treatment support apparatus according to claim 9, wherein
the interface circuit is connectable to a measurement device for measuring a vital sign of the patient, and
the vital sign information is acquired from the measurement device.

11. The medical treatment support apparatus according to claim 8, wherein
the interface circuit is connectable to a medication device for automatically administering the medicine to the patient, and
the processor executes the program to:
determine a dose of the medicine to be administered to the patient based on the determined test value, and
control the medication device to administer the determined dose of the medicine to the patient.

12. The medical treatment support apparatus according to claim 8, wherein
the medicine is an anticoagulant or an antiplatelet agent.

13. The medical treatment support apparatus according to claim 8, wherein
the one or more values include a pump rotation speed or a blood flow rate of the extracorporeal blood circulation device.

14. The medical treatment support apparatus according to claim 8, wherein
the determined test value is activated clotting time (ACT) or activated partial thromboplastin time (APTT).

15. A medical treatment support apparatus comprising:
a display device;
an interface circuit connectable to an extracorporeal blood circulation device;
a memory that stores a program; and
a processor configured to execute the program to:
acquire a plurality of pieces of administration information each related to administration of a medicine to a patient,
acquire a plurality of pieces of device operation information each including a value related to an operation of the extracorporeal blood circulation device,
determine a plurality of combinations of the pieces of administration information and the pieces of device operation information,
execute one or more calls to a machine learning model with each of the determined combinations to determine a plurality of test values that are expected from a blood test performed on the patient at a predetermined time after the medicine of the corresponding dose is administered, the machine learning model having been trained with:
a plurality of pieces of administration information regarding administration of the medicine to different patients,
a plurality of pieces of device operation information used by the extracorporeal blood circulation device for treating the different patients, and
test values obtained before and after administration of the medicine to the different patients,
generate a screen showing at least one of:
a graph showing either a relationship between the pieces of administration information and the determined test values or a relationship between the pieces of device operation information and the determined test values, and
a table of the pieces of administration information and the determined test values or the pieces of device operation information and the determined test values, and
control the display device to display the generated screen.

16. The medical treatment support apparatus according to claim 15, wherein
the machine learning model has been further trained using physical information and vital sign information relating to the different patients, and
the processor executes the program to:
acquire physical information and vital sign information relating to the patient, and
execute the calls to the machine learning model further using the physical information and the vital sign information.

17. The medical treatment support apparatus according to claim 16, wherein
the interface circuit is connectable to a measurement device for measuring a vital sign of the patient, and
the vital sign information is acquired from the measurement device.

18. The medical treatment support apparatus according to claim 15, wherein
the processor executes the program to determine at least one of a dose of the medicine to be administered to the patient and a value related to the operation and to be used for treating the patient based on the determined test values.

19. The medical treatment support apparatus according to claim 15, wherein
the processor executes the program to:
classify the determined test values into a plurality of classes using one or more threshold values, and associate each of the pieces of administration information with one of the classes into which the corresponding test value is classified, and
output text indicating the pieces of administration information and the corresponding classes on the screen.

20. The medical treatment support apparatus according to claim 15, wherein
the values included in the pieces of device operation information include at least one of: an upper limit value that can be set to the extracorporeal blood circulation device for the operation, a lower limit value that can be set to the extracorporeal blood circulation device for the operation, and a median value between the upper and lower limit values.
